(19) United States
(12) Patent Application Publication
(10) Pub. No.: US 2003/0153861 A1
(43) Pub. Date: Aug. 14, 2003

(54) Wound treatment bandage

(76) Inventor: George R. Royer, Toledo, OH (US)

Correspondence Address: George R. Royer, 2137 Ragan Woods Drive, Toledo, OH, 43614, US

(21) Appl. No.: 10/072,229

(22) Filed: Feb. 11, 2002

(51) Int. Cl. A61F 13/00

(52) U.S. Cl. 602/48; 602/54

(57) ABSTRACT

The subject invention is an improved band aid (bandage covering) for use in treatment of wounds where medication is to be administered to a surface wound, infection, or other problem on the skin of an individual, such band aid comprising, in general, a bandage member or similar member adapted to cover the skin area where the wound is located, such bandage member having a portion thereof with an opening on the upper bandage surface that extends to the lower surface of the bandage and which opening has a removable cover over the upper part thereof with the opening being used to insert medication to reach a specific wound area under the bandage and which opening is then recovered after the medication is so placed.

Priority of filing is based On Applicants provisional application serial No. 60-306393 filed Jul. 20, 2001.

### C. KNOWN PRIOR ART

None.

### D. DISCUSSION OF PRIOR ART AND BACKGROUND OF INVENTION

The subject invention is a bandage member similar to a band aid which is adapted to cover a wound, infected area, or sore on a person's skin. This invention is considered to be a refinement or improvement on the existing status of such band aid member.

As is well known, band aids are used to cover a cut, wound or even some infected area on the skin to protect that area from further germs or other exacerbations of the problem area. These bandage members are well known and effective to cover a wounded area from exacerbation or further problems, and are generally effective for such purposes. Moreover, in the more recent evolutions, the undersurface of the band aid may be coated with an antibiotic as a further protective measure for the wound.

However, not all skin surface problems are just wounds, as many problems may include skin infections such as warts, psoraisis, plant poison reactions, general surface infections not caused by skin wounds, and numerous other problems, including skin cancers. For these types of skin problems, it is common to use non-prescription ointments such as salves, or creams with some antibiotic properties, or other healing agents. Additionally, for more severe skin conditions, prescription drugs in the form of cream applications or salves, or external medicinal applications are prescribed and used.

A large percentage of these skin problems are usually localized in a given skin area, sometimes being as small as a few centimeters in diameter or less, while other are a little larger. Some skin disorders may be widespread and covering large areas of the skin over which area some medicine needs to be applied.

As to the localized and smaller areas of skin disorders to which medicine is to be applied, once the prescribed medication is applied to the specific skin area, there is no general or effective means to keep the medication in place for the necessary duration to treat the skin area. For example, the nature of the substances used for medical treatment results in the substance spreading beyond the skin problem area to areas that are not infected or affected by the problem. The spreading or movement of the treating medication can be aggravated by body movements, and the medication, depending on its location, can be picked up by and absorbed by clothing or other contact. In short, when the medication is applied to a localized problem skin area, there is no effective means to keep medication in place in sufficiently concentrated amounts to adequately treat the area. Applying a band aid over the medicated area helps to some degree but using such could result only in more speading or absorption of the medication in a large surface area of the band aid.

The problem discussed above is not simply a problem of losing the effectiveness of concentrated amounts of a necessary medication. There is the additional problem that such medications often have some deleterious and adverse effect on non-infected skin areas, and the problem of having the medication spreading to non-infected areas may result in skin damage to such areas, particularly when the applied medication has caustic or other dangerous side effects to the skin. Under such latter circumstances, it is important and vital to keep the medication localized to the prlblem area as much as possible.

Since there are no known means to effectively prevent this problem of medication straying or spreading from the intended infected area, this invention is conceived as a result and the following objects of the subject invention are directed accordingly.

### D. OBJECTS

It is an object of the subject invention to provide an improved band aid device;

A further object of the subject invention is to provide a novel bandage device for concentrating medicinal applications to limited skin areas;

Yet another object of the subject invention is to provide an improved health protection device;

A further object of the subject invention is to provide an improved bandage to help prevent the spread of skin infections;

Still another object of the subject invention is to provide an improved skin protective device;

A further object of the subject invention is to provide a band aid to aid in the concentrated application of medicine;

Yet another object of the subject invention is to provide an improved device to aid in the application of skin medication;

Other and further objects of the subject invention will become apparent from a reading of the following description taken in conjunction with the claims.

### E. DRAWINGS

FIG. 1 is a side elevational view of the device incorporating the subject invention, showing the medicinal container in the open position;

FIG. 2 is a top elevational view of the apparatus incorporating the subject invention;

FIG. 3 is a side elevational view of the apparatus incorporating the subject invention, showing the medicinal container in the closed position;

FIG. 4 is a top elevational view of the subject invention showing the medicinal container in the closed position.

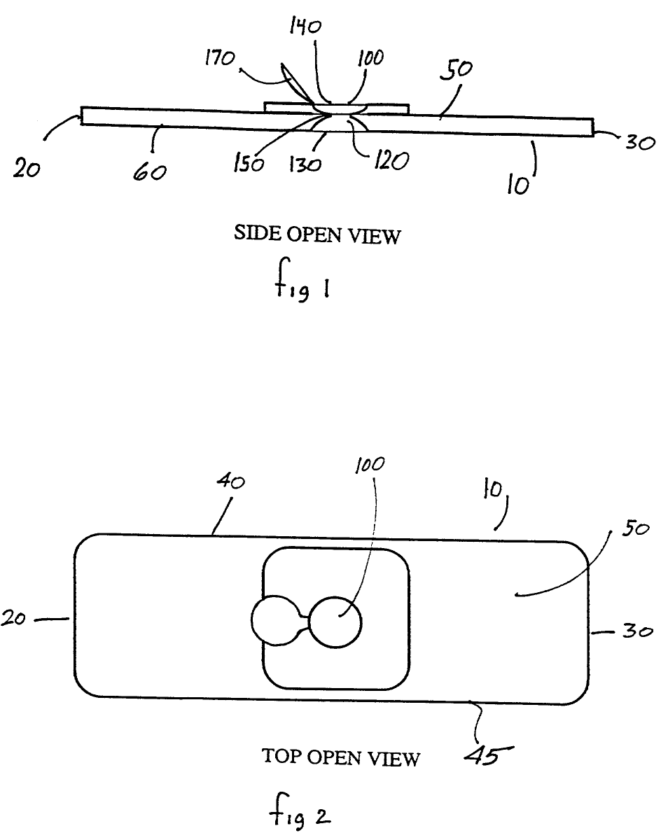

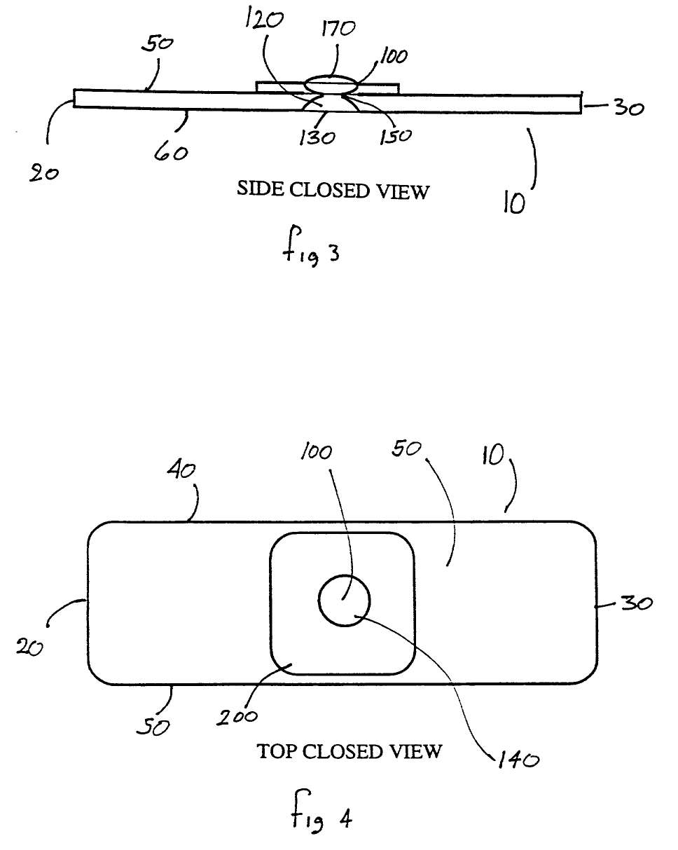

### F. DESCRIPTION OF GENERAL EMBODIMENT

The subject invention is an improved Band Aid(®) (bandage covering) for use in treatment of wounds where medication is to be administered to a surface wound, infection, or other problem on the skin of an individual, such band aid comprising, in general, a bandage member or similar member adapted to cover the skin area where the wound is located, such bandage member having a portion thereof with an opening on the upper bandage surface that extends to the lower surface of the bandage and which opening has a removable cover over the upper part thereof with the opening being used to insert medication to reach a specific wound area under the bandage and which opening is then recovered after the mediciation is so placed.

In general and in summary, the subject invention is a band aid type bandage member that is basically structured as a conventional band aid having an upper surface comprised of some protective covering material and a lower surface part of which has an adhesive coating to adhere the member to the skin, and part of which lower surface has a bandage covering for placement over the skin wound. In the structural adaptation covered herein, the band aid member has an opening that extends downwardly from the upper surface to and through the lower surface. This opening is adapted to be positioned over a wound or infected area of the skin. Once the band aid member is placed on the skin with the opening being placed over the particular infected area of the skin, a medicinal application, whether it be liquid, or a viscous cream, or whatever is placed into the opening at the upper surface of the band aid to permit the medicinal substance to seep downwardly through the opening to the bottom of the opening at the lower surface to reach the infected area.

In the general embodiment therein a cover flap will be appended to the upper surface of the band aid member to cover the opening once the medicine is placed in the opening. This cover will obviously function to retain medicine in the opening to seep downwardly over the wound. Thus, once the band aid member is affirxed in place the cover flap is placed and secured over the opening, the medicinal substance will be limited to the opening area over the infected skin area. This will keep the medicine only over the infected area and will prevent it from spreading to non-infected areas or simply falling off the skin.

### G. DESCRIPTION OF PREFERRED EMBODIMENT

In describing the preferred embodiment, it is to be stressed that the following description is of only one embodiment within the overall scope of the subject invention and that the subject invention may encompass other embodiments, as seen in the claims annexed hereto. Therefore, the following description shall not be considered as limiting the scope of the invention herein, as more fully set forth in the claims annexed hereto.

Referring now to the drawings in which a preferred embodiment of the subject invention is shown. A band aid 10 is shown as being the device incorporating the features of the subject invention, such device 10 being basically comprised of a longitudinally extending band aid member 10 having a first end 20 and a second end 30, and a first lateral side edge 40 and a second lateral side edge 45. Such band aid has an upper surface 50 and a lower surface 60. The upper surface 50 of the band aid member is comprised of a protective material such as plastic, fabric or other material to shield the skin from germs, moisture or other substances that would interfere with the healing of the skin or infected area on the skin. The lower surface 60 of the band aid has areas covered with adhesive substances to help adhere the band aid 10 to the specific areas of the skin. It is noted that in this particular embodiment of the invention, it may be preferable to have the entire lower surface 60 of the band aid coated with adhesive material, as an option to having some bandage material in the middle portion. By this arrangement, there will be adhesive materials surrounding the bottom of the opening in the band aid to keep the lower surface 60 sealed against the skin to block the radially outer movement of applied medication away from the wound or infected area. This aspect is more fully discussed below.

As thus far described above, the band aid structure is generally conventional. In the preferred embodiment of the subject invention, an upper entrance opening 100 is formed in the upper surface 50 of the band aid 10, preferably but not essentially circular, which opening extends completely through the band aid from such upper surface to bottom surface 60 to a lower exit opening on the lower surface. This opening may have as an optional feature a conformingly-shaped cup 120, which is seated concentrically and securely in the opening 100 so that the cup 120 is held firmly within the opening. As seen, the cup 120 would have a bottom opening 130 and a top opening 140 into which medicine can be poured to be held temporarily in the cup to seep down through a narrowed throat 150 in the cup to the bottom opening 130. The bottom opening would preferably be large enough to encircle the wound or infected area. The upper opening of the cup 120 would have a closure lid 170 which would be fastened in place after the medicine is placed on the wound.

Alternately, the opening 100 may not have a cup therein, and medication could simply be placed in the opening to cover the particular area of the skin involved. In one embodiment of this latter structure, the upper opening may have a cover flap 200 to be placed flush against the opening and sealed at the other end to the upper portion of the band aid.

It is to be noted that band aids of different sizes with varying opening sizes may be provided, depending on the size of the affected skin area. Other embodiments are contemplated as being within the scope of this invention, and in this respect the subject invention is not limited to the shapes and sizes described.

1. An improved band aid member having an upper surface and a lower surface, such band aid having means to aid in the application and retention of medication on an area of a person's skin comprising:
(a) a band aid member having an upper surface and a lower surface, and member having an opening extending from the upper surface to and through the lower surface, said opening being adapted to receive medication for the given area of the person's skin;
(b) adhesive means coated in the lower surface of the band aid member.
